(19) United States
(12) Patent Application Publication
(10) Pub. No.: US 2025/0355351 A1
(43) Pub. Date: Nov. 20, 2025

(54) PHOTOSENSITIVE RESIN COMPOSITION, PHOTOSENSITIVE RESIN LAYER USING THE SAME, DISPLAY DEVICE, AND MANUFACTURING METHOD OF PHOTOSENSITIVE RESIN LAYER

(71) Applicant: Samsung SDI Co., Ltd., Yongin-si (KR)

(72) Inventors: Heejo MOON, Suwon-si (KR); Baek Soung PARK, Suwon-si (KR); Kwangwon SEO, Suwon-si (KR); Ickjin KIM, Suwon-si (KR); Subin PARK, Suwon-si (KR); Youngmin JEON, Suwon-si (KR)

(21) Appl. No.: 18/967,970

(22) Filed: Dec. 4, 2024

(30) Foreign Application Priority Data
May 20, 2024 (KR) 10-2024-0065466

(51) Int. Cl. G03F 7/031; C07D 251/24; G03F 7/00; G03F 7/033; G03F 7/16; H10K 59/38; H10K 59/80

(52) U.S. Cl. CPC G03F 7/031; C07D 251/24; G03F 7/0007; G03F 7/033; G03F 7/168; G03F 7/70008; H10K 59/38; H10K 59/873

(57) ABSTRACT

A photosensitive resin composition, a photosensitive resin layer manufactured using the photosensitive resin composition, a display device including the photosensitive resin layer, and a method of manufacturing the photosensitive resin layer, the photosensitive resin composition including a binder resin; a photopolymerizable monomer; a photopolymerization initiator, and a solvent, wherein the wherein the photopolymerizable monomer is represented by Chemical Formula 1,

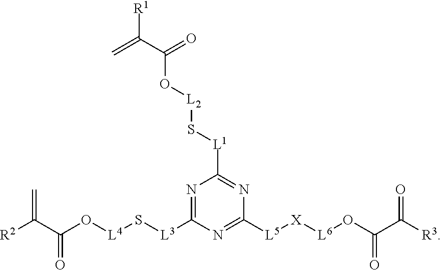

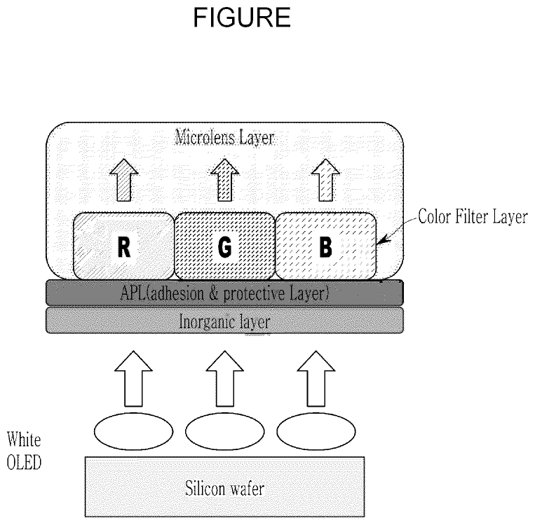

### CROSS-REFERENCE TO RELATED APPLICATION

This application claims priority to and the benefit of Korean Patent Application No. 10-2024-0065466 filed in the Korean Intellectual Property Office on May 20, 2024, the entire contents of which are incorporated herein by reference.

### BACKGROUND

### 1 Field

Embodiments relate to a photosensitive resin composition, a photosensitive resin layer using the same, a display device, and a method of manufacturing the photosensitive resin layer.

### 2. Description of the Related Art

Recently, interest in self-emitting (emissive) micro OLED display panels, which may be applied to VR (Virtual Reality), AR (Augmented Reality), and MR (Mixed Reality) devices, has increased.

### SUMMARY

The embodiments may be realized by providing a photosensitive resin composition, including a binder resin; a photopolymerizable monomer; a photopolymerization initiator; and a solvent, wherein the photopolymerizable monomer is represented by Chemical Formula 1,

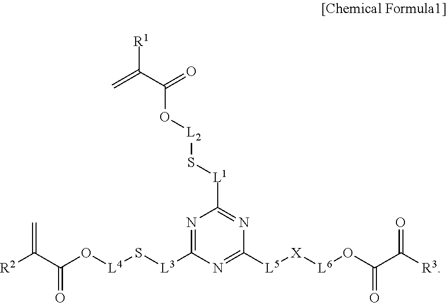

in Chemical Formula 1, R¹ to R³ are each independently a hydrogen atom or a substituted or unsubstituted C1 to C20 alkyl group, L¹ to L⁴ are each independently a substituted or unsubstituted C6 to C20 arylene group, L⁵ is a single bond or a substituted or unsubstituted C6 to C20 arylene group, L⁶ is a substituted or unsubstituted C1 to C20 alkylene group or a substituted or unsubstituted C6 to C20 arylene group, X is a single bond, *—S—* or *—NR′—*, and R′ is a hydrogen atom or a C1 to C10 alkyl group.
L¹ to L⁴ may all be the same.
R¹ to R³ may all be the same.
L⁵ may be a substituted or unsubstituted C6 to C20 arylene group, and X may be a single bond or *—S—*.
L⁵ may be a single bond and X may be *—S—* or *—NR′—*, and R′ may be a hydrogen atom or a C1 to C10 alkyl group.
L⁵ may be a single bond and X may be a single bond.

The photopolymerizable monomer may represented by one of Chemical Formula 1-1 to Chemical Formula 1-5:

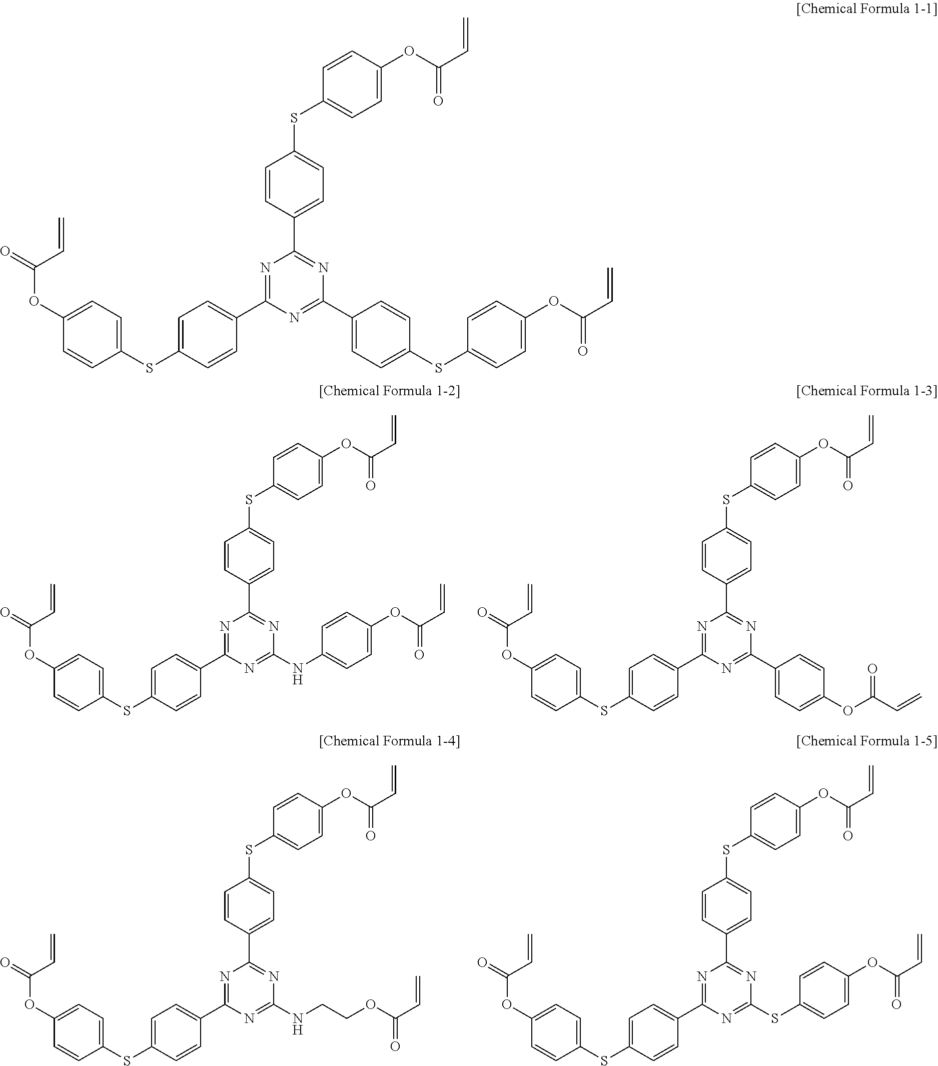

The photosensitive resin composition may include, based on a total weight of the photosensitive resin composition, about 10 wt % to about 30 wt % of the binder resin; about 3 wt % to about 15 wt % of the photopolymerizable monomer; and about 0.1 wt % to about 5 wt % of the photopolymerization initiator.

The photosensitive resin composition may further include malonic acid, 3-amino-1,2-propanediol, a silane coupling agent, a leveling agent, a surfactant, a polymerization inhibitor, or a combination thereof.

The photosensitive resin composition may have a refractive index of greater than or equal to about 1.66 at 550 nm.

The photosensitive resin composition may have a transmittance of greater than or equal to about 90% at 400 nm to 700 nm.

The embodiments may be realized by providing a photosensitive resin layer manufactured using the photosensitive resin composition according to some embodiments.

The embodiments may be realized by providing a display device including the photosensitive resin layer according to some embodiments.

The display device may be a micro OLED display device including an OLED substrate on a silicon wafer and a color filter layer located on the OLED substrate, the color filter layer being configured to convert white light generated from the OLED substrate into a plurality of color lights, the photosensitive resin layer may be located on the OLED substrate and color filter layer, and the color filter layer may include a red color filter, a green color filter, and a blue color filter.

The embodiments may be realized by providing a method of manufacturing a photosensitive resin layer, the method including coating the photosensitive resin composition according to an embodiment on a substrate; prebaking at a temperature of about 100° C. or lower after coating; exposing to i-line after prebaking, and developing after exposing.

### BRIEF DESCRIPTION OF THE DRAWING

Features will become apparent to those of skill in the art by describing in detail exemplary embodiments with reference to the attached drawing in which:

the FIGURE is a schematic diagram showing the structure of a micro OLED display device according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when specific definition is not otherwise provided, “alkyl group” refers to a C1 to C20 alkyl group, “alkenyl group” refers to a C2 to C20 alkenyl group, “cycloalkenyl group” refers to a C3 to C20 cycloalkenyl group, “heterocycloalkenyl group” refers to a C3 to C20 heterocycloalkenyl group, “aryl group” refers to a C6 to C20 aryl group, “arylalkyl group” refers to a C6 to C20 arylalkyl group, “alkylene group” refers to a C1 to C20 alkylene group, “arylene group” refers to a C6 to C20 arylene group, “alkylarylene group” refers to a C6 to C20 alkylarylene group, “heteroarylene group” refers to a C3 to C20 heteroarylene group, and “alkoxylene group” refers to a C1 to C20 alkoxylene group.

As used herein, when specific definition is not otherwise provided, “substituted” refers to replacement of at least one hydrogen of a compound by a substituent selected from a halogen atom (F, Cl, Br, or I), a hydroxy group, a C1 to C20 alkoxy group, a nitro group, a cyano group, an amine group, an imino group, an azido group, an amidino group, a hydrazino group, a hydrazono group, a carbonyl group, a carbamyl group, a thiol group, an ester group, an ether group, a carboxyl group or a salt thereof, a sulfonic acid group or a salt thereof, a phosphoric acid or a salt thereof, a C1 to C20 alkyl group, a C2 to C20 alkenyl group, a C2 to C20 alkynyl group, a C6 to C20 aryl group, a C3 to C20 cycloalkyl group, a C3 to C20 cycloalkenyl group, a C3 to C20 cycloalkynyl group, a C2 to C20 heterocycloalkyl group, a C2 to C20 heterocycloalkenyl group, a C2 to C20 heterocycloalkynyl group, a C3 to C20 heteroaryl group, or a combination thereof.

As used herein, when specific definition is not otherwise provided, “hetero” refers to inclusion of at least one heteroatom of N, O, S, and P, in the chemical formula.

As used herein, when a specific definition is not otherwise provided, “(meth)acrylate” refers to both “acrylate” and “methacrylate”, and “(meth)acrylic acid” refers to “acrylic acid” and “methacrylic acid”.

As used herein, when a definition is not otherwise provided, the term “combination” refers to mixing or copolymerization. Additionally, “copolymerization” refers to block copolymerization to random copolymerization, and “copolymer” refers to block copolymerization to random copolymerization.

In the chemical formula of the present specification, unless a specific definition is otherwise provided, hydrogen is bonded at the position when a chemical bond is not drawn where supposed to be given. As used herein, hydrogen substitution (—H) may include deuterium substitution (-D) or tritium substitution (-T). For example, any hydrogen in any compound described herein may be protium, deuterium, or tritium (e.g., based on natural or artificial substitution).

As used herein, when a definition is not otherwise provided, “*” refers to a linking part between the same or different atoms, or chemical formulas.

A photosensitive resin composition according to some example embodiments may include (A) a binder resin; (B) a photopolymerizable monomer represented by Chemical Formula 1; (C) a photopolymerization initiator; and (D) a solvent.

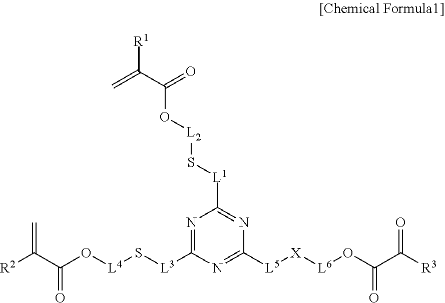

In Chemical Formula 1, R¹ to R³ may each independently be or include, e.g., a hydrogen atom or a substituted or unsubstituted C1 to C20 alkyl group.
L¹ to L⁴ may each independently be, e.g., a substituted or unsubstituted C6 to C20 arylene group.
L⁵ may be, e.g., a single bond or a substituted or unsubstituted C6 to C20 arylene group.
L⁶ may be, e.g., a substituted or unsubstituted C1 to C20 alkylene group or a substituted or unsubstituted C6 to C20 arylene group.
X may be, e.g., a single bond, *—S—*, or *—NR′—*, wherein R′ may be, e.g., a hydrogen atom or a C1 to C10 alkyl group.

A liquid crystal display device is a type of display that may have an advantage of lightness, thinness, low cost, low power consumption for operation, and improved adhesion to an integrated circuit and has been widely used in laptop computers, monitors, and TV screens. The liquid crystal display device may include a lower substrate on which a black matrix, a color filter, and an ITO pixel electrode are formed, and an upper substrate on which an active circuit portion including a liquid crystal layer, a thin film transistor, and a capacitor layer, and an ITO pixel electrode are formed. Color filters may be formed in a pixel region by sequentially stacking a plurality of color filters (e.g., formed of a plurality of colors, e.g., formed of red (R), green (G), and blue (B)) in a predetermined order to form each pixel, and a black matrix layer may be in a predetermined pattern on a transparent substrate to form a boundary between the pixels. The pigment dispersion method may be a method of forming a color filter and may provide a colored thin film by repeating a series of processes, e.g., coating a photopolymerizable composition including a colorant on a transparent substrate including a black matrix, exposing a formed pattern to light, removing a non-exposed part with a solvent, and thermally curing the same. A color photosensitive resin composition used for manufacturing a color filter according to the pigment dispersion method may include an alkali soluble resin, a photopolymerization monomer, a photopolymerization initiator, an epoxy resin, a solvent, other additives, or the like and additionally, an epoxy resin or the like. The pigment dispersion method having the above characteristics may be applied to manufacture of an LCD for use in, e.g., a mobile phone, a laptop, a monitor, or a TV.

However, there may be limitations in terms of resolution, if applying the technology of manufacturing a color filter for liquid crystal displays to VR and AR devices, which have recently attracted lots of attention in the market. In order to implement high resolutions of greater than or equal to about 4000 ppi, OLEDoS (OLED on Silicon) technology is being introduced, which is technology using OLEDs deposited on a silicon wafer as backlights and patterning a color filter thereon. While some color filters used for liquid crystal displays may be cured through a post-baking process at a high temperature of greater than or equal to about 230° C., a color filter mounted on OLEDoS must be curable at a low temperature due to durability of OLED materials. In addition, micropatterning may be essential to achieve desired resolution within a small size of the VR and AR devices. This technology, in which curing may be possible only at a low temperature (e.g., about 100° C.), may have color changes due to low chemical resistance. To avoid color changes, the hardness of a resist may be increased.

Displays like OLED may have low efficiency if light generated therefrom leaks out. To avoid unwanted light leakage, a high refractive index layer or a high refractive index pattern may be used to control a refractive index difference, which is one of the causes of light loss when the light leaks.

As an attempt to make a polymer compound that is highly functional, development of polyimide polymer materials including sulfur atoms has been made to secure a high refractive index (>1.60). However, such polyimide materials may have properties of absorbing light in a wavelength range of around 400 nm and thus may not be suitable for microlens that receive light in the visible light region (about 400 nm to about 700 nm).

Present embodiments relate not to a color photoresist which may be used for manufacturing a color filter but to the microlens applied to micro OLED display devices, wherein the microlens may maintain a high refractive index at 550 nm of greater than or equal to about 1.66, e.g., greater than or equal to about 1.67, greater than or equal to about 1.68, greater than or equal to about 1.69, or greater than or equal to about 1.7, but still have high transmittance in the visible light region of greater than or equal to about 90% or, e.g., greater than or equal to about 95%, and thus may be very suitable for receiving light.

Hereinafter, each component is described in detail.

### (A) Binder Resin

The binder resin may include an acrylic binder resin. The acrylic binder resin may be, e.g., a copolymer of a first ethylenically unsaturated monomer and a second ethylenically unsaturated monomer copolymerizable therewith, and may be, e.g., a resin including one or more acrylic repeating units.

The first ethylenically unsaturated monomer may be, e.g., an ethylenically unsaturated monomer including, e.g., a carboxyl group. Examples thereof may include, e.g., acrylic acid, methacrylic acid, maleic acid, itaconic acid, fumaric acid, or a combination thereof.

The first ethylenically unsaturated monomer may be included, e.g., in an amount of about 5 wt % to about 50 wt %, e.g., about 10 wt % to about 40 wt %, based on a total weight of the acrylic binder resin.

Examples of the second ethylenic unsaturated monomer may include, e.g., an aromatic vinyl compound, e.g., styrene, α-methylstyrene, vinyltoluene, vinylbenzylmethylether, or the like; an unsaturated carboxylic acid ester compound, e.g., methyl(meth)acrylate, ethyl(meth)acrylate, butyl(meth)acrylate, 2-hydroxyethyl(meth)acrylate, 2-hydroxy butyl(meth)acrylate, benzyl(meth)acrylate, cyclohexyl(meth)acrylate, phenyl(meth)acrylate, or the like; an unsaturated carboxylic acid amino alkyl ester compound, e.g., 2-aminoethyl(meth)acrylate, 2-dimethylaminoethyl(meth)acrylate, or the like; a carboxylic acid vinyl ester compound, e.g., vinyl acetate, vinyl benzoate, or the like; an unsaturated carboxylic acid glycidyl ester compound, e.g., glycidyl(meth)acrylate or the like; a vinyl cyanide compound, e.g., (meth)acrylonitrile or the like; an unsaturated amide compound, e.g., (meth)acrylamide or the like; or the like, and may be used alone or as a mixture of two or more.

Examples of the acrylic binder resin may include, e.g., a (meth)acrylic acid/benzylmethacrylate copolymer, a (meth)acrylic acid/benzylmethacrylate/styrene copolymer, a (meth)acrylic acid/benzylmethacrylate/2-hydroxyethylmethacrylate copolymer, a (meth)acrylic acid/benzylmethacrylate/styrene/2-hydroxyethylmethacrylate copolymer or the like, and may be used alone or as a mixture of two or more.

The acrylic binder resin may have a weight average molecular weight of about 3,000 g/mol to about 20,000 g/mol and a double bond equivalent of greater than or equal to about 340 g/mol. Maintaining the weight average molecular weight and double bond equivalent of the acrylic binder resin within the above ranges may help ensure excellent pattern forming properties, and that the manufactured thin film can have excellent mechanical and thermal properties.

The binder resin may include an epoxy binder resin. The binder resin may improve heat resistance by further including an epoxy binder resin. The epoxy binder resin may be, e.g., a phenol novolac epoxy resin, a tetramethyl biphenyl epoxy resin, a bisphenol A epoxy resin, a bisphenol F epoxy resin, an alicyclic epoxy resin, or a combination thereof.

In an implementation, the binder resin including the epoxy binder resin may help secure dispersion stability of a colorant such as a pigment, described below, and may help form a pixel having a desired resolution during a developing process.

The epoxy binder resin may be included in an amount of about 1 wt % to about 10 wt %, e.g., about 5 wt % to about 10 wt %, based on a total weight of the binder resin. Maintaining the amount of the epoxy binder resin within the above ranges may help ensure that the film residue ratio and chemical resistance may be greatly improved.

An epoxy equivalent weight of the epoxy resin may be about 150 g/eq to about 200 g/eq. Including an epoxy binder resin having an epoxy equivalent within the above range in the binder resin may help ensure that there is an advantageous effect in improving a curing degree of the formed pattern and fixing the colorant in the structure in which the pattern is formed.

In an implementation, the binder resin may be included in an amount of about 10 wt % to about 30 wt %, e.g., about 10 wt % to about 25 wt %, based on a total weight of the photosensitive resin composition. Maintaining the binder resin within the above ranges may help ensure that excellent sensitivity, developability, resolution, and linearity of the pattern may be obtained.

### (B) Photopolymerizable Monomer

The photopolymerizable monomer in the photosensitive resin composition according to some example embodiments may be, e.g., a single compound or a mixture of two different types of compounds.

In an implementation, the photopolymerizable monomer may be, e.g., a trifunctional ester of (meth)acrylic acid having at least one ethylenically unsaturated double bond, and, in an implementation, the photopolymerizable monomer may be represented by Chemical Formula 1.

If the photopolymerizable monomer is represented by Chemical Formula 1, it can form a pattern having excellent heat resistance, light resistance, and chemical resistance by causing sufficient polymerization upon exposure in the pattern forming process, and can also have a high refractive index at 550 nm and high transmittance in the visible light region.

In an implementation, all of L¹ to L⁴ may be, e.g., the same and all of L¹ to L⁴ may be, e.g., a substituted or unsubstituted C6 arylene group, e.g., a phenylene group.

In an implementation, all of R¹ to R³ may be the same, and all of R¹ to R³ may be, e.g., a methyl group or a hydrogen atom.

In an implementation, L⁵ may be, e.g., a substituted or unsubstituted C6 to C20 arylene group and X may be, e.g., a single bond or *—S—*.

In an implementation, L⁵ may be, e.g., a single bond and X may be, e.g., *—S—* or *—NR′—*, wherein R′ may be, e.g., a hydrogen atom or a C1 to C10 alkyl group.

In an implementation, L⁵ may be, e.g., a single bond and X may be, e.g., a single bond.

In an implementation, the photopolymerizable monomer may be represented by one of Chemical Formula 1-1 to Chemical Formula 1-5.

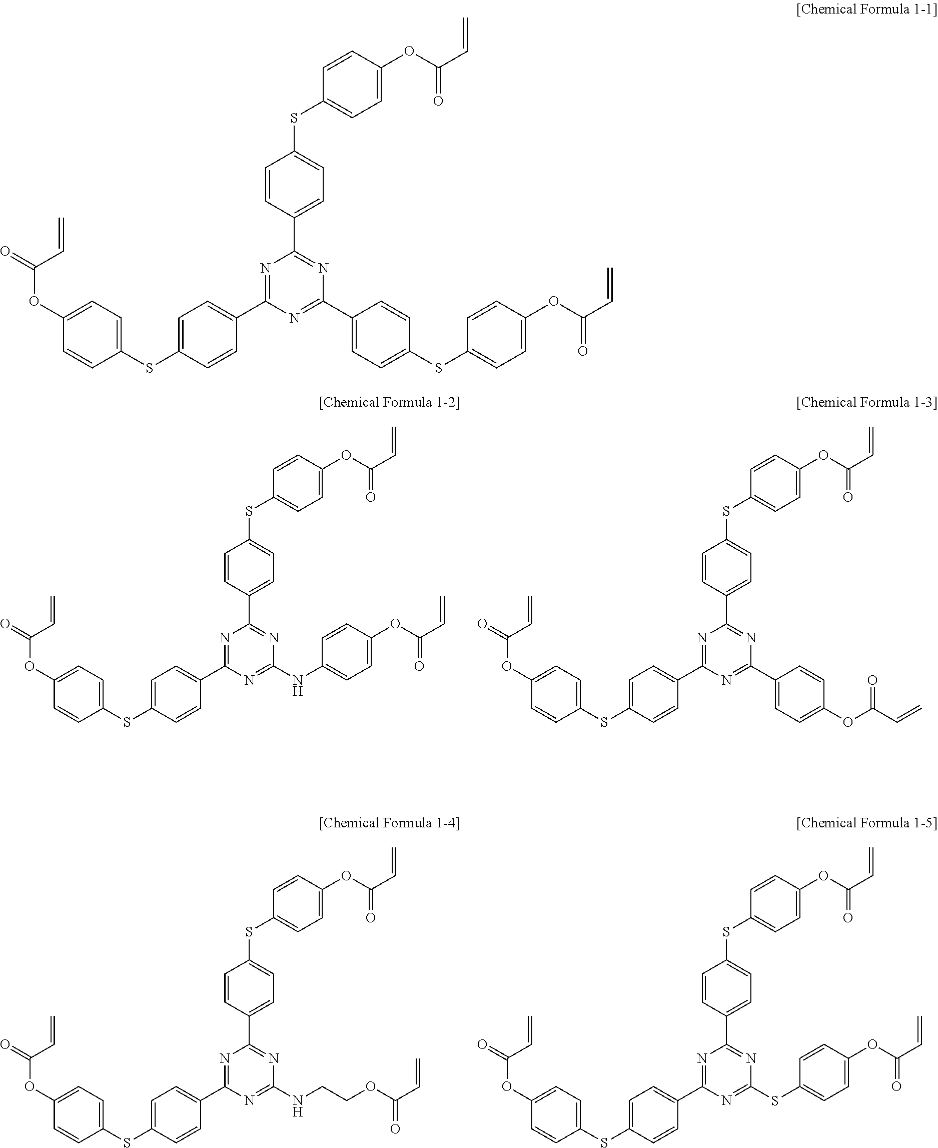

In an implementation, the photosensitive resin composition may further include. e.g., a photopolymerizable monomer having a different structure than Chemical Formula 1 together with the photopolymerizable monomer represented by Chemical Formula 1.

In an implementation, the photopolymerizable monomer having a structure different from Chemical Formula 1 may be, e.g., ethylene glycol di(meth)acrylate, diethylene glycol di(meth)acrylate, triethylene glycol di(meth)acrylate, propylene glycol di(meth)acrylate, neopentyl glycol di(meth)acrylate, 1,4-butanediol di(meth)acrylate, 1,6-hexanediol di(meth)acrylate, bisphenol A di(meth)acrylate, pentaerythritol di(meth)acrylate, pentaerythritol tri(meth)acrylate, pentaerythritol tetra(meth)acrylate, pentaerythritol hexa(meth)acrylate, dipentaerythritol di(meth)acrylate, dipentaerythritol tri(meth)acrylate, dipentaerythritol penta(meth)acrylate, dipentaerythritol hexa(meth)acrylate, bisphenol A epoxy(meth)acrylate, ethylene glycol monomethylether (meth)acrylate, trimethylol propane tri(meth)acrylate, tris(meth)acryloyloxyethyl phosphate, novolac epoxy(meth)acrylate, ethoxylated bisphenyl fluorene diacrylate, ethoxylated naphthalene type diacrylate, ethoxylated sulfur type diacrylate, or the like.

Commercially available examples of the photopolymerizable monomer having a structure different from Chemical Formula 1 are as follows. Examples of the mono-functional ester of (meth)acrylic acid may include, e.g., Aronix M-101®, M-111®, M-114® (Toagosei Chemistry Industry Co., Ltd.); KAYARAD TC-110S®, TC-120S® (Nippon Kayaku Co., Ltd.); V-158®, V-2311® (Osaka Organic Chemical Ind., Ltd.), or the like. Examples of a difunctional (meth)acrylic acid ester may include, e.g., Aronix M-210®, M-240®, M-6200® (Toagosei Chemistry Industry Co., Ltd.), KAYARAD HDDA®, HX-220®, R-604® (Nippon Kayaku Co., Ltd.), V-260®, V-312®, V-335 HP® (Osaka Organic Chemical Ind., Ltd.), BPF-022, BPF-022B, BPF-022L, BPF-022G, BPF-102, BPF-152, BPF-202, BPF-302, BN-042, BN-102, TBP-042, TBP-102 (Hannong Chemicals Inc.) or the like. Examples of a tri-functional ester of (meth)acrylic acid may include, e.g., Aronix M-309®, M-400®, M-405®, M-450®, M-710®, M-8030®, M-8060® (Toagosei Chemistry Industry Co., Ltd.), KAYARAD TMPTA®, DPCA-20®, DPCA-30®, DPCA-60®, DPCA-120® (Nippon Kayaku Co., Ltd.), V-295®, V-300®, V-360®, V-GPT®, V-3PA®, V-400® (Osaka Yuki Kayaku Kogyo Co. Ltd.), or the like. These may be used alone or as a mixture of two or more.

The photopolymerizable monomer may be treated with acid anhydride to improve developability. In an implementation, the photopolymerizable monomer may be included in an amount of about 3 wt % to about 15 wt %, e.g., about 5 wt % to about 10 wt %, based on a total weight of the photosensitive resin composition. In an implementation, the photopolymerizable monomer may be included in an amount of about 20 wt % to about 60 wt %, e.g., about 30 wt % to about 50 wt %, based on a total weight of solids constituting the photosensitive resin composition. Including the photopolymerizable monomer within these ranges may help ensure that the photopolymerizable monomer may be sufficiently cured during exposure in a pattern-forming process and has excellent reliability, and that developability for an alkali developer may be improved.

### (C) Photopolymerization Initiator

The photosensitive resin composition according to some example embodiments may include a photopolymerization initiator. The photopolymerization initiator may include, e.g., an acetophenone compound, a benzophenone compound, a thioxanthone compound, a benzoin compound, a triazine compound, an oxime compound, or the like.

Examples of the acetophenone compound may include, e.g., 2,2′-diethoxy acetophenone, 2,2′-dibutoxy acetophenone, 2-hydroxy-2-methylpropinophenone, p-t-butyltrichloro acetophenone, p-t-butyldichloro acetophenone, 4-chloroacetophenone, 2,2′-dichloro-4-phenoxy acetophenone, 2-methyl-1-(4-(methylthio)phenyl)-2-morpholinopropan-1-one, 2-benzyl-2-dimethylamino-1-(4-morpholinophenyl)-butan-1-one, or the like.

Examples of the benzophenone compound may include, e.g., benzophenone, benzoyl benzoate, benzoyl methyl benzoate, 4-phenyl benzophenone, hydroxy benzophenone, acrylated benzophenone, 4,4′-bis(dimethyl amino)benzophenone, 4,4′-bis(diethylamino)benzophenone, 4,4′-dimethylaminobenzophenone, 4,4′-dichlorobenzophenone, 3,3′-dimethyl-2-methoxybenzophenone, or the like.

Examples of the thioxanthone compound may include, e.g., thioxanthone, 2-chlorothioxanthone, 2-methylthioxanthone, isopropyl thioxanthone, 2,4-diethyl thioxanthone, 2,4-diisopropyl thioxanthone, or the like.

Examples of the benzoin compound may include, e.g., benzoin, benzoin methyl ether, benzoin ethyl ether, benzoin isopropyl ether, benzoin isobutyl ether, benzyl dimethyl ketal, or the like.

Examples of the triazine compound may include, e.g., 2,4,6-trichloro-s-triazine, 2-phenyl 4,6-bis(trichloromethyl)-s-triazine, 2-(3′,4′-dimethoxystyryl)-4,6-bis(trichloromethyl)-s-triazine, 2-(4′-methoxynaphthyl)-4,6-bis(trichloromethyl)-s-triazine, 2-(p-methoxyphenyl)-4,6-bis(trichloromethyl)-s-triazine, 2-(p-tolyl)-4,6-bis(trichloro methyl)-s-triazine, 2-biphenyl 4,6-bis(trichloromethyl)-s-triazine, bis(trichloromethyl)-6-styryl-s-triazine, 2-(naphthol-yl)-4,6-bis(trichloromethyl)-s-triazine, 2-(4-methoxynaphthol-yl)-4,6-bis(trichloromethyl)-s-triazine, 2-4-bis(trichloromethyl)-6-piperonyl-s-triazine, 2-4-bis(trichloromethyl)-6-(4-methoxystyryl)-s-triazine, or the like.

Examples of the oxime compound may include, e.g., an O-acyloxime compound, 2-(O-benzoyloxime)-1-[4-(phenylthio)phenyl]-1,2-octanedione, or 1-(O-acetyloxime)-1-[9-ethyl-6-(2-methylbenzoyl)-9H-carbazol-3-yl]ethenone. In an implementation, the o-acyloxime compound may be, e.g., 1,2-octanedione, 2-dimethylamino-2-(4-methylbenzyl)-1-(4-morpholin-4-yl-phenyl)-butan-1-one, 1-(4-phenylsulfanyl phenyl)-butane-1,2-dione-2-oxime-O-benzoate, 1-(4-phenylsulfanyl phenyl)-octane-1,2-dione2-oxime-O-benzoate, 1-(4-phenylsulfanyl phenyl)-octan-1-oneoxime-O-acetate, 1-(4-phenylsulfanyl phenyl)-butan-1-one, oxime-O-acetate, or the like.

In an implementation, the photopolymerization initiator may also be used together with, e.g., a carbazole compound, a diketone compound, a sulfonium borate compound, a diazo compound, an imidazole compound, a biimidazole compound, or the like.

In an implementation, the photopolymerization initiator may be included in an amount of about 0.1 wt % to about 5 wt %, e.g., about 0.5 wt % to about 3 wt %, based on a total weight of the photosensitive resin composition. In an implementation, the photopolymerization initiator may be included in an amount of about 1 wt % to about 5 wt %, e.g., about 1.5 wt % to about 3 wt %, based on a total weight of solids constituting the photosensitive resin composition. Including the photopolymerization initiator within the above range may help ensure that photopolymerization occurs sufficiently during exposure in the pattern formation process for manufacturing microlenses, resulting in excellent sensitivity and improved transmittance.

### (D) Solvent

The solvent may be, e.g., a material that is compatible with, but does not react with, the polymer resin, the photopolymerizable monomer, and the photopolymerization initiator.

Examples of the solvent may include, e.g., alcohols such as methanol, ethanol, and the like; ethers such as dichloroethylether, n-butylether, diisoamylether, methylphenylether, tetrahydrofuran, or the like; glycolethers such as ethylene glycolmonomethylether, ethylene glycolmonoethylether, ethylene glycoldimethylether, or the like; cellosolveacetates such as methylcellosolveacetate, ethylcellosolveacetate, diethylcellosolveacetate, or the like; carbitols such as methylethylcarbitol, diethylcarbitol, diethylene glycolmonomethylether, diethylene glycolmonoethylether, diethylene glycoldimethylether, diethylene glycolethylmethylether, diethylene glycoldiethylether, or the like; propylene glycolalkyletheracetates such as propylene glycolmethyletheracetate, propylene glycolpropyletheracetate, or the like; aromatic hydrocarbons such as toluene, xylene, or the like; ketones such as methylethylketone, cyclohexanone, 4-hydroxy-4-methyl-2-pentanone, methyl-n-propylketone, methyl-n-butylketone, methyl-n-amylketone, 2-heptanone, or the like; saturated aliphatic monocarboxylic acid alkyl esters such as ethyl acetate, n-butyl acetate, isobutyl acetate, or the like; lactate esters such as methyl lactate, ethyl lactate, or the like; oxy acetic acid alkyl esters such as oxy methyl acetate, oxy ethyl acetate, oxy butyl acetate, or the like; alkoxy acetic acid alkyl esters such as methoxy methyl acetate, methoxy ethyl acetate, methoxy butyl acetate, ethoxy methyl acetate, ethoxy ethyl acetate, or the like; 3-oxypropionic acid alkyl esters such as 3-oxymethyl propionate, 3-oxyethyl propionate, or the like; 3-alkoxypropionic acid alkyl esters such as 3-methoxymethyl propionate, 3-methoxyethyl propionate, 3-ethoxyethyl propionate, 3-ethoxymethyl propionate, or the like; 2-oxypropionic acid alkyl esters such as 2-oxymethyl propionate, 2-oxyethyl propionate, 2-oxypropyl propionate, or the like; 2-alkoxypropionic acid alkyl esters such as 2-methoxymethyl propionate, 2-methoxyethyl propionate, 2-ethoxyethyl propionate, 2-ethoxymethyl propionate, or the like; 2-oxy-2-methylpropionic acid esters such as 2-oxy-2-methylmethyl propionate, 2-oxy-2-methylethyl propionate, or the like, monooxy monocarboxylic acid alkyl esters of 2-alkoxy-2-methyl alkyl propionates such as 2-methoxy-2-methylmethyl propionate, 2-ethoxy-2-methylethyl propionate, or the like; esters such as 2-hydroxyethyl propionate, 2-hydroxy-2-methylethyl propionate, hydroxy ethyl acetate, 2-hydroxy-3-methyl methyl butanoate, or the like; ketonate esters such as ethyl pyruvate, and the like. In an implementation, a high boiling point solvent such as N-methylformamide, N,N-dimethyl formamide, N-methylformanilide, N-methylacetamide, N,N-dimethyl acetamide, N-methylpyrrolidone, dimethylsulfoxide, benzylethylether, dihexylether, acetylacetone, isophorone, caproic acid, caprylic acid, 1-octanol, 1-nonanol, benzyl alcohol, benzyl acetate, ethyl benzoate, diethyl oxalate, diethyl maleate, γ-butyrolactone, ethylene carbonate, propylene carbonate, phenyl cellosolve acetate, or the like may be also used.

In an implementation, considering miscibility and reactivity, glycolethers such as ethylene glycolmonoethylether, ethylene glycoldimethylether, ethylene glycoldiethylether, diethylene glycolethylmethylether, or the like; ethylene glycolalkylether acetates such as ethyl cellosolveacetate, or the like; esters such as 2-hydroxyethyl propionate, or the like; carbitols such as diethylene glycolmonomethylether, or the like; propylene glycolalkyletheracetates such as propylene glycolmonomethyl ether acetate, propylene glycolpropyletheracetate, or the like may be used.

The solvent may be included in a balance amount, e.g., about 50 wt % to about 90 wt %, about 60 wt % to about 80 wt %, about 70 wt % to about 80 wt %, based on a total weight of the photosensitive resin composition. Maintaining the solvent within the above ranges may help ensure that the photosensitive resin composition may have an appropriate viscosity and thus processability may be improved during a production of a photosensitive resin layer, specifically, a microlens.

### (E) Other Additives

In an implementation, the photosensitive resin composition may further include additives, e.g., malonic acid, 3-amino-1,2-propanediol, a silane coupling agent, a leveling agent, a surfactant, a polymerization inhibitor, or a combination thereof.

The silane coupling agent may have a reactive substituent, e.g., a vinyl group, a carboxyl group, a methacryloxy group, an isocyanate group, or an epoxy group to improve adhesion to the substrate. If a silane coupling agent is used, because the photosensitive resin composition according to some example embodiments may be a colorant-free composition, e.g., a transparent photosensitive resin composition, if the refractive index of the polymer resin at 550 nm is not controlled as described above, an effect of improving adhesion to the color filter may not be obtained.

Examples of the silane coupling agent may include, e.g., trimethoxysilylbenzoic acid, γ-methacryloxypropyltrimethoxysilane, vinyltriacetoxysilane, vinyltrimethoxysilane, γ-isocyanatepropyltriethoxysilane, γ-glycidoxypropyltrimethoxysilane, β-(3,4-epoxycyclohexyl)ethyltrimethoxysilane, and the like. These may be used alone or in a mixture of two or more.

The silane coupling agent may be included in an amount of about 0.01 parts by weight to about 10 parts by weight, based on 100 parts by weight of the photosensitive resin composition. Including the silane coupling agent within the above range, adhesion, storage capability, or the like may be improved.

In an implementation, the photosensitive resin composition may further include a surfactant, e.g., a fluorine surfactant or a silicone surfactant to improve coating properties and prevent defect formation.

Examples of the fluorine surfactant may include a commercially available fluorine surfactant such as BM-1000®, BM-1100®, or the like of BM Chemie Inc.; MEGAFACE F 142D®, MEGAFACE F 172®, MEGAFACE F 173®, MEGAFACE F 183®, MEGAFACE F 554®, or the like of Dainippon Ink Kagaku Kogyo Co., Ltd.; FULORAD FC-135®, FULORAD FC-170C®, FULORAD FC-430®, FULORAD FC-431®, or the like of SUMITOMO 3M Co., Ltd.; SURFLON S-112®, SURFLON S-113®, SURFLON S-131®, SURFLON S-141®, SURFLON S-145®, or the like of Asahi Glass Co., Ltd.; SH-28PAR, SH-190®, SH-193®, SZ-6032®, SF-8428®, or the like of Toray Silicone Co., Ltd.

The surfactant may include a commercial silicone surfactant such as BYK-307, BYK-333, BYK-361N, BYK-051, BYK-052, BYK-053, BYK-067A, BYK-077, BYK-301, BYK-322, BYK-325, or the like of BYK Chem.

The surfactant may be included in an amount of about 0.001 parts by weight to about 5 parts by weight, based on 100 parts by weight of the photosensitive resin composition. Including the surfactant within the above range may help ensure that coating uniformity may be secured, a stain may not be produced, and wetting on an IZO substrate or a glass substrate is improved.

In an implementation, the polymerization inhibitor may include a catechol compound. In an implementation, the photosensitive resin composition according to some example embodiments may further include the catechol compound and room temperature crosslinking may be prevented during exposure to light after coating the photosensitive resin composition.

In an implementation, the catechol compound may include, e.g., catechol, t-butyl catechol, 4-methoxyphenol, pyrogallol, 2,6-di-t-butyl-4-methylphenol, 2-naphthol, tris(N-hydroxy-N-nitrosophenylaminato-O,O′)aluminum.

The catechol compound may be included, e.g., in the form of a dispersion, and the polymerization inhibitor in the form of the dispersion may be included in an amount of about 0.001 wt % to about 3 wt %, e.g., about 0.01 wt % to about 1 wt %, based on a total weight of the photosensitive resin composition. Maintaining the polymerization inhibitor within the above range may help ensure that it is possible to solve the problem of aging at room temperature and prevent deterioration of sensitivity and surface peeling.

In an implementation, the photosensitive resin composition may include other additives e.g., a stabilizer, or the like, in a predetermined amount unless they deteriorate properties of the photosensitive resin composition.

The photosensitive resin composition according to some example embodiments may be either positive or negative. In an implementation, the composition may be negative to completely remove residues in regions where a pattern is exposed after exposing and developing the composition having light blocking properties.

In an implementation, the photosensitive resin composition according to some example embodiments, which includes the photopolymerizable monomer represented by Chemical Formula 1, may have a refractive index of greater than or equal to about 1.66 (at about 550 nm), e.g., greater than or equal to about 1.62 and less than or equal to about 1.9 (at about 550 nm). In an implementation, the photosensitive resin composition according to some example embodiments, which includes a photopolymerizable monomer represented by Chemical Formula 1, may have transmittance of greater than or equal to about 90%, e.g., greater than or equal to about 95%, greater than or equal to about 96%, or greater than or equal to about 97% in the visible light region (about 400 nm to about 700 nm). The photosensitive resin composition including the photopolymerizable monomer represented by Chemical Formula 1, of which the refractive index at about 550 nm and the transmittance in the visible light region are controlled as described above, may significantly reduce the light loss, if white light generated from an OLED substrate leaks out and may further advantageously contribute to improving developability and fine patternability. Because of these characteristics, the photosensitive resin composition according to some example embodiments may be very suitable as a microlens material for OLEDoS.

Some example embodiments may provide a photosensitive resin layer manufactured by low-temperature curing, exposure, and development of the aforementioned photosensitive resin composition. Compared to other LCD or semiconductor processes, the difference may be that the post-curing (post-baking) process may be unnecessary.

A method of manufacturing the photosensitive resin layer may be as follows.

### (1) Coating and Film Formation (Low-Temperature Curing)

The photosensitive resin composition may be coated to a desired thickness on a substrate such as a silicon wafer, or the like which may undergo a predetermined pretreatment, using a spin or slit coating method, a roll coating method, a screen-printing method, an applicator method, or the like, and may be heated at about 100° C. for about 1 minute to 10 minutes to remove a solvent and may thereby form a photosensitive resin layer. Through this step, it may be possible to improve image quality, or the like.

### (2) Exposure

After disposing a mask to form a necessary pattern on the obtained photosensitive resin layer, exposure may be performed by irradiating an actinic ray of i-line. As a light source used for irradiation, a low-pressure mercury lamp, a high-pressure mercury lamp, an ultra-high pressure mercury lamp, a metal halide lamp, an argon gas laser, or the like may be used, and in some cases, an X-ray, an electron beam, or the like may be used.

The exposure process may use, e.g., a light dose of 500 mJ/cm² or less (with a 365 nm sensor) if a high-pressure mercury lamp is used. The light dose may vary depending on the types of each component, a combination ratio thereof, and a dry film thickness. Through this step, fine adjustment of the pixel size may be possible, enabling high-resolution implementation.

### (3) Development

In the development method, following the exposure step, an alkaline aqueous solution may be used as a developer to dissolve and remove unnecessary parts, leaving only the exposed parts remaining to form a pattern. Through this step, a profile can be formed to realize high resolution, and color uniformity can be secured to improve clarity.

Some example embodiments may provide a display device including the photosensitive resin layer.

The display device may be a micro organic light emitting diode (OLED) display device.

The micro organic light emitting diode (OLED) display device may include an OLED substrate deposited on a silicon wafer and a color filter layer located on the OLED substrate and converting white light generated from the OLED substrate into a plurality of color lights. In an implementation, the color filter layer may include a red color filter, a green color filter, and a blue color filter. In an implementation, the photosensitive resin layer, i.e., the microlens layer, may be located on the OLED substrate and color filter layer. In an implementation, the microlens layer may surround the color filter layer on the OLED substrate.

In an implementation, the micro organic light emitting diode (OLED) display device may include an OLED substrate on a silicon wafer, an inorganic layer stacked on the OLED substrate, a color filter layer stacked on the inorganic layer and converting white light generated from the OLED substrate into a plurality of color lights, and the color filter layer may include a red color filter, a green color filter, and a blue color filter. In an implementation, the photosensitive resin layer, i.e., the microlens layer, may be located on the inorganic layer and the color filter layer. In an implementation, the microlens layer may surround the color filter layer on the inorganic layer.

Referring to the FIGURE, an implementation, the micro organic light emitting diode (OLED) display device may include an OLED substrate on a silicon wafer, an inorganic layer stacked on the OLED substrate, an adhesive protection layer, i.e., adhesion & protective layer, stacked on the inorganic layer, and a color filter layer stacked on the adhesive protection layer and converting white light generated from the OLED substrate into a plurality of color lights. In an implementation, the color filter layer may include a red color filter, a green color filter, and a blue color filter. In an implementation, the photosensitive resin layer, i.e., the microlens layer, may be located on the adhesive protection layer and the color filter layer. In an implementation, the microlens layer may surround the color filter layer on the adhesive protection layer.

On some OLED substrates OLEDs may be deposited on a glass or polyimide substrate. The micro OLED display device according to some example embodiments may be more advantageous in implementing a micro display because the OLED may be deposited on a silicon wafer. These micro displays are in the spotlight as next-generation displays, and the micro displays are expected to be applied to devices such as MR.

The micro OLED display device with the above structure can be driven on a pixel basis by depositing WOLED on a highly integrated silicon wafer, and it may be easy to control the transmission wavelength through a color filter layer patterned with a resolution of less than or equal to about 3 μm, enabling high color reproduction and securing high resolution.

In an implementation, the adhesive protection layer may have a thickness of less than or equal to 1 μm. In this case, the above-mentioned effects, that is, the effects of improving adhesion to the color filter and improving residue characteristics, may be further maximized.

In an implementation, the color filter layer may have a thickness of about 1.1 μm to about 1.6 μm.

Maintaining the thickness of the color filter layer within the above range may help ensure that it may be more advantageous to implement in a micro OLED display device.

In an implementation, the inorganic layer may have a thickness of less than or equal to about 2 μm. Even with WOLED, light may not always diffuse in the direction perpendicular to the OLED substrate, so that color mixing of red, green, and blue could occur. Therefore, an inorganic layer could be deposited on the OLED substrate to prevent such color mixing. The color mixing may not be completely prevented even by depositing an inorganic layer and, in some example embodiments, subtle light leakage phenomenon can be prevented by thinning the inorganic layer, e.g., controlling the thickness of the inorganic layer to less than or equal to about 2 μm.

In an implementation, the photosensitive resin composition according to some example embodiments may allow the production of a cured layer only through low-temperature (100° C.) curing during prebaking and i-line photocuring, as described above, and there may be a huge difference in resolution that can be implemented compared to other display devices.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### EXAMPLES

### Preparation Example 1: Synthesis of Compound Represented by Chemical Formula 1-1

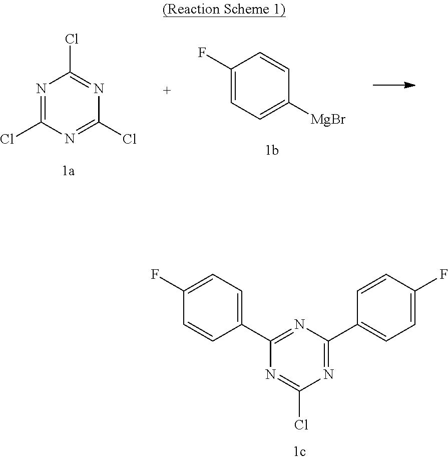

The compound 1a (10.0 mmol) was dissolved in 20 mL of THF (tetrahydrofuran) to obtain a solution, and after cooling the solution to 0° C., another solution prepared by dissolving the compound 1b (22.0 mmol) in THF was added thereto in a dropwise fashion. After the dropwise addition, the obtained mixture was stirred at ambient temperature overnight. After a reaction was completed, a 1 N HCl aqueous solution was added thereto and then extracted by using ethyl acetate. An organic layer obtained therefrom was anhydrous-treated, concentrated under a reduced pressure, and then purified through column chromatography (eluent: n-hexane/EtOAc) to obtain the compound 1c.

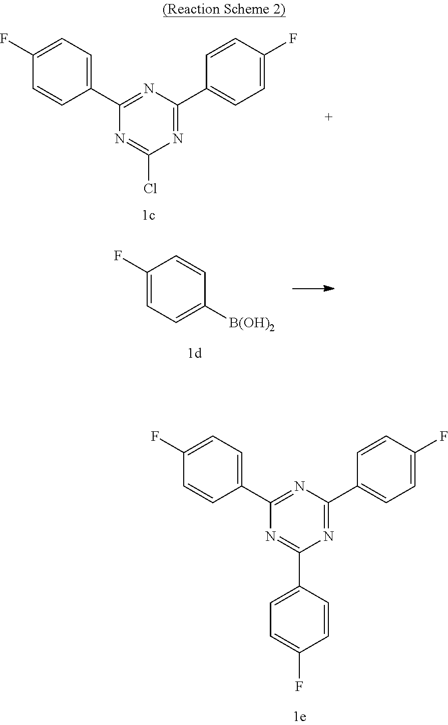 [SMILES: Fc1ccc(-c2nc(Cl)nc(-c3ccc(F)cc3)n2)cc1 ; OB(O)c1ccc(F)cc1 ; Fc1ccc(-c2nc(-c3ccc(F)cc3)nc(-c3ccc(F)cc3)n2)cc1]

The compound 1c (10.0 mmol) and the compound 1d (12.0 mmol) were added with Pd(PPh₃)₄ (0.30 mmol) and potassium carbonate (K₂CO₃, 50.0 mmol) to 80.0 mL of toluene/H₂O (v/v=5/1) and reacted under reflux overnight. After cooling to ambient temperature, an organic layer was separated therefrom and then concentrated under a reduced pressure to remove the solvent. Subsequently, methanol was added to the reaction mixture to produce precipitates, which were separated by filtering under a reduced pressure and then washed with distilled water. The compound 1e was obtained by drying overnight in a vacuum oven.

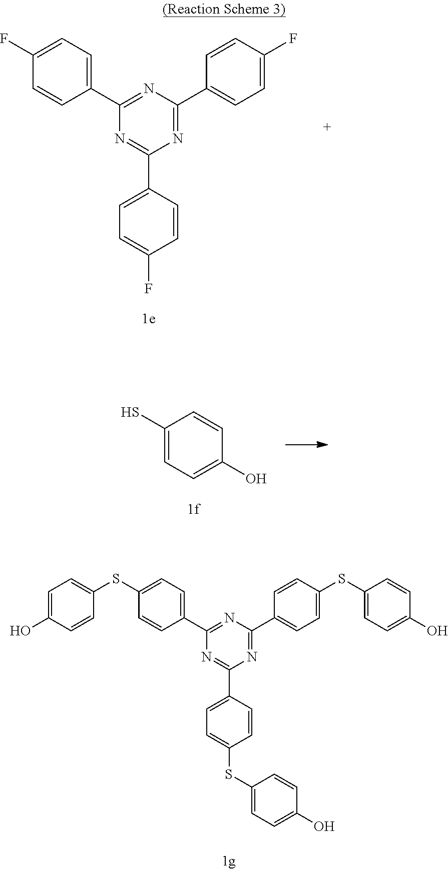 [SMILES: Fc1ccc(-c2nc(-c3ccc(F)cc3)nc(-c3ccc(F)cc3)n2)cc1 ; Oc1ccc(S)cc1 ; Oc1ccc(Sc2ccc(-c3nc(-c4ccc(Sc5ccc(O)cc5)cc4)nc(-c4ccc(Sc5ccc(O)cc5)cc4)n3)cc2)cc1]

The compound 1e (10.0 mmol) and the compound 1f (60.0 mmol) were added with potassium carbonate (K₂CO₃, 60.0 mmol) to 35.0 mL of DMF (N,N-dimethylformamide) and then reacted under reflux overnight. After cooling to ambient temperature, the resultant was added to 100 mL of a 1 N HCl aqueous solution to produce precipitates. The solids were separated by filtering under a reduced pressure, washed with distilled water, and dried overnight in a vacuum oven. Subsequently, the compound 1g was obtained by purification through column chromatography (eluent: n-hexane/EtOAc).

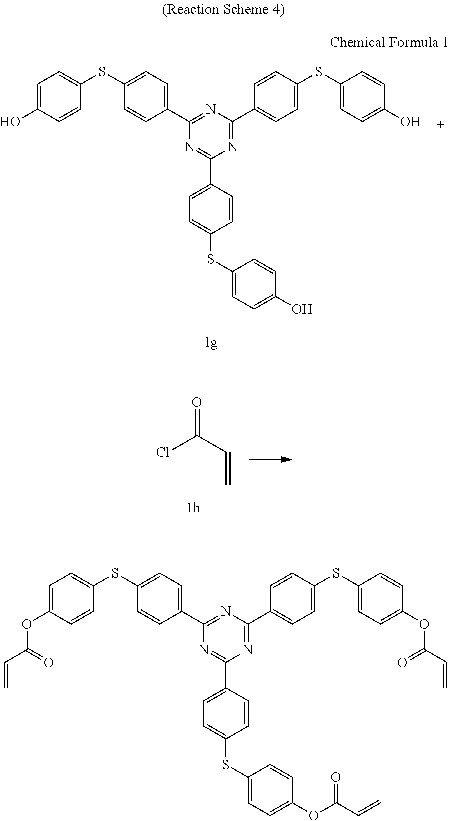

The compound 1g (5.00 mmol) and triethylamine (30.0 mmol) were dissolved in 30.0 mL of THF (tetrahydrofuran), and after cooling the solution to 0° C., another solution prepared by dissolving the compound 1h (22.5 mmol) in 10.0 mL of THF was slowly added thereto. The reaction mixture was stirred at ambient temperature overnight, and a saturated sodium bicarbonate aqueous solution was added thereto. The obtained solution was diluted by using distilled water and then extracted by using DCM (dichloromethane). An organic layer therefrom was passed through MgSO₄, concentrated under a reduced pressure, and purified through column chromatography (eluent: DCM) to obtain a compound represented by Chemical Formula 1.

The analysis result of the compound represented by Chemical Formula 1 is as follows.

MALDI-TOF MS: 844.15 m/z

### Preparation Example 2: Synthesis of Compound Represented by Chemical Formula 2

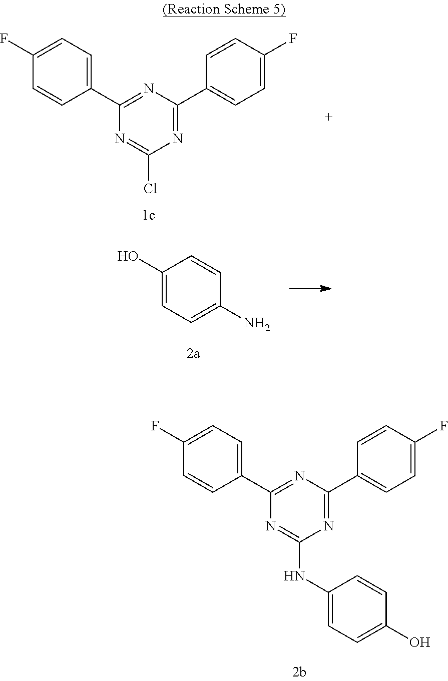 [SMILES: Fc1ccc(-c2nc(Cl)nc(-c3ccc(F)cc3)n2)cc1 ; Nc1ccc(O)cc1 ; Oc1ccc(Nc2nc(-c3ccc(F)cc3)nc(-c3ccc(F)cc3)n2)cc1]

The compound 1c (5.00 mmol) and DIPEA (N,N-diisopropylethylamine, 7.50 mmol) were dissolved in 15.0 mL of THF (tetrahydrofuran), and the compound 2a (6.00 mmol) was added to the solution. The obtained mixture was stirred at ambient temperature overnight. After removing the solvent therefrom by concentration under a reduced pressure, the compound 2b was obtained therefrom by purification through column chromatography (eluent: n-hexane/EtOAc).

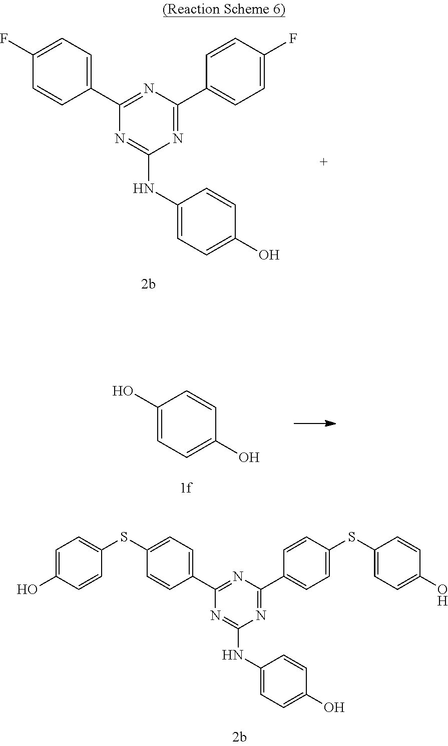 [SMILES: Oc1ccc(Nc2nc(-c3ccc(F)cc3)nc(-c3ccc(F)cc3)n2)cc1 ; Oc1ccc(O)cc1 ; Oc1ccc(Nc2nc(-c3ccc(Sc4ccc(O)cc4)cc3)nc(-c3ccc(Sc4ccc(O)cc4)cc3)n2)cc1]

The compound 2b (10.0 mmol) and the compound 1f (40.0 mmol) were added with potassium carbonate (K₂CO₃, 40.0 mmol) to 35.0 mL of DMF (N,N-dimethylformamide) and then reacted under reflux overnight. After cooling to ambient temperature, the resultant was added to 100 mL of a 1 N HCl aqueous solution to produce precipitates. The solids were separated by filtering under a reduced pressure, washed with distilled water, and dried in a vacuum oven overnight. The compound 2c was obtained by purification through column chromatography (eluent: n-hexane/EtOAc).

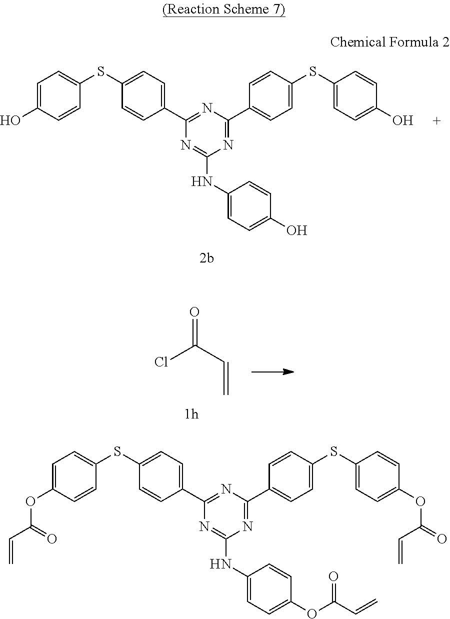

A compound represented by Chemical Formula 2 was synthesized in the same manner as the method of synthesizing the material corresponding to Chemical Formula 1 except that the compound 2c was used instead of the compound 1g.

The analysis results of the compound represented by Chemical Formula 2 are as follows.

MALDI-TOF MS: 751.16 m/z

### Preparation Example 3: Synthesis of Compound Represented by Chemical Formula 3

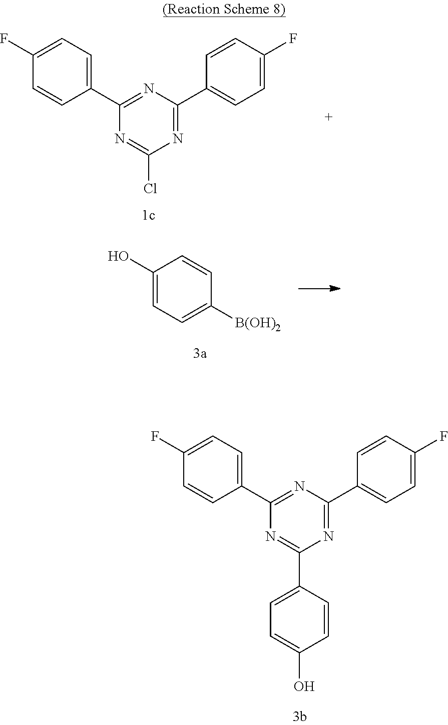 [SMILES: Fc1ccc(-c2nc(Cl)nc(-c3ccc(F)cc3)n2)cc1 ; OB(O)c1ccc(O)cc1 ; Oc1ccc(-c2nc(-c3ccc(F)cc3)nc(-c3ccc(F)cc3)n2)cc1]

The compound 3b was synthesized in the same manner as the method of synthesizing the compound 1e, except that the compound 3a was used instead of the compound 1d.

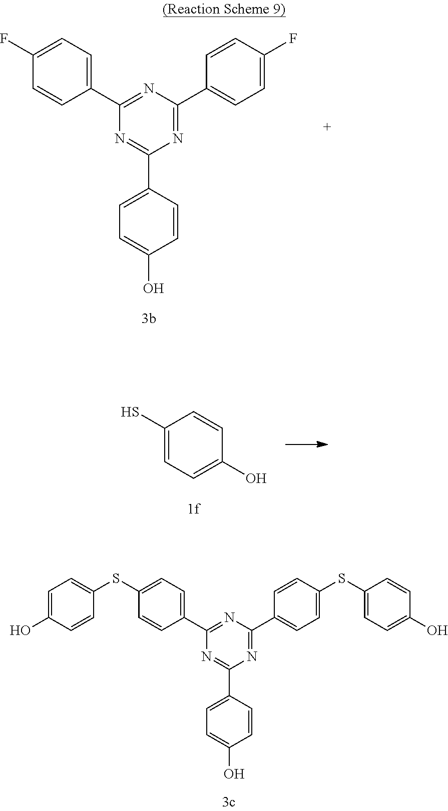 [SMILES: Oc1ccc(-c2nc(-c3ccc(F)cc3)nc(-c3ccc(F)cc3)n2)cc1 ; Oc1ccc(S)cc1 ; Oc1ccc(Sc2ccc(-c3nc(-c4ccc(O)cc4)nc(-c4ccc(Sc5ccc(O)cc5)cc4)n3)cc2)cc1]

The compound 3c was synthesized in the same manner as the method of synthesizing the compound 2c except that the compound 3b was used instead of the compound 2b.

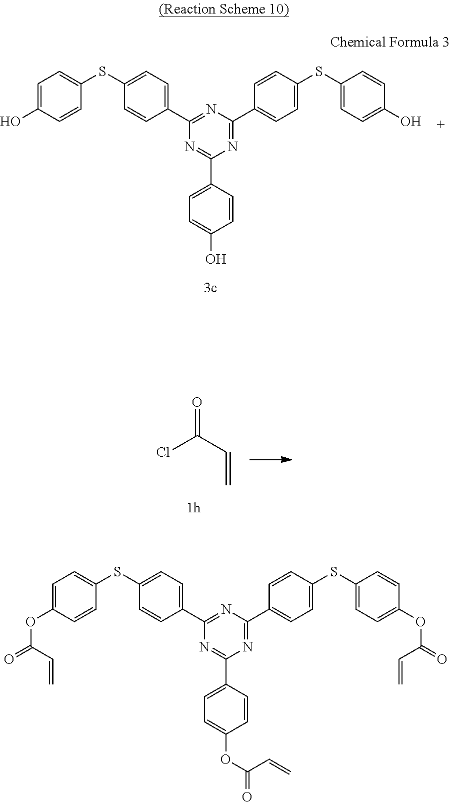

A compound represented by Chemical Formula 3 was synthesized in the same manner as the method of synthesizing the material corresponding to Chemical Formula 1 except that the compound 3c was used instead of the compound 1g.

The analysis result of the compound represented by Chemical Formula 3 is as follows.

MALDI-TOF MS: 736.15 m/z

### Preparation Example 4: Synthesis of Compound Represented by Chemical Formula 4

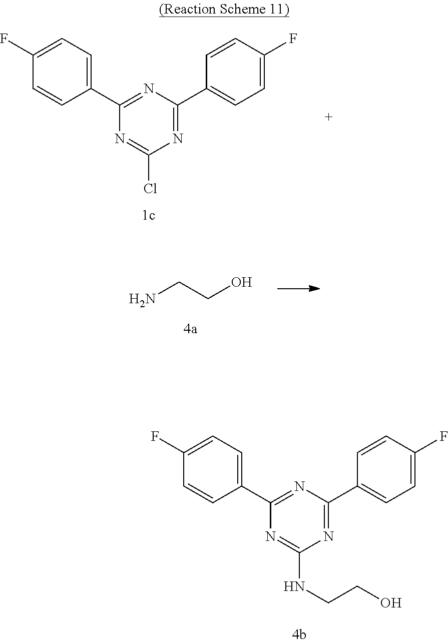

The compound 4b was synthesized in the same manner as the method of synthesizing the compound 2b except that the compound 4a was used instead of the compound 2a.

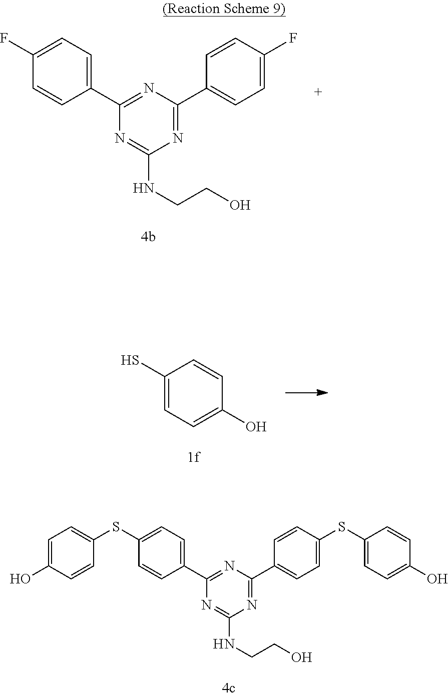 [SMILES: OCCNc1nc(-c2ccc(F)cc2)nc(-c2ccc(F)cc2)n1 ; Oc1ccc(S)cc1 ; OCCNc1nc(-c2ccc(Sc3ccc(O)cc3)cc2)nc(-c2ccc(Sc3ccc(O)cc3)cc2)n1]

The compound 4c was synthesized in the same manner as the method of synthesizing the compound 2c except that the compound 4b was used instead of the compound 2b.

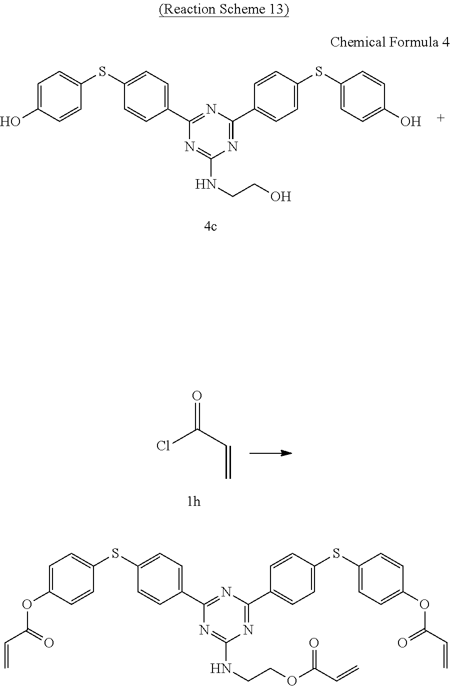

A compound represented by Chemical Formula 4 was synthesized in the same manner as the method of synthesizing the material corresponding to Chemical Formula 1 except that the compound 4c was used instead of the compound 1g.

The analysis result of the compound represented by Chemical Formula 4 is as follows.

MALDI-TOF MS: 703.16 m/z

### Preparation Example 5: Synthesis of Compound Represented by Chemical Formula 5

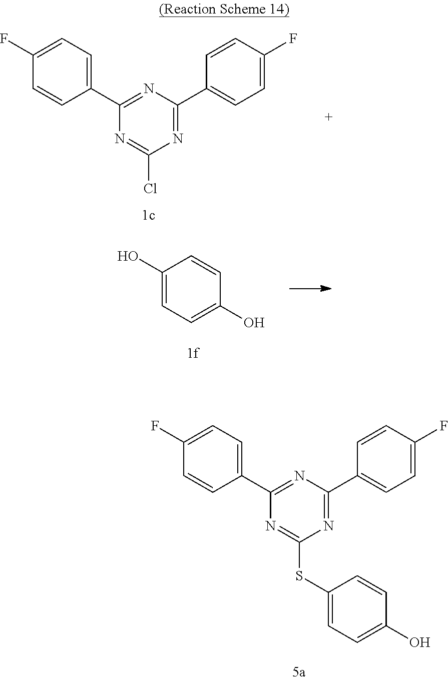 [SMILES: Fc1ccc(-c2nc(Cl)nc(-c3ccc(F)cc3)n2)cc1 ; Oc1ccc(O)cc1 ; Oc1ccc(Sc2nc(-c3ccc(F)cc3)nc(-c3ccc(F)cc3)n2)cc1]

The compound 5a was synthesized in the same manner as the method of synthesizing the compound 2b except that the compound 1f was used instead of the compound 2a.

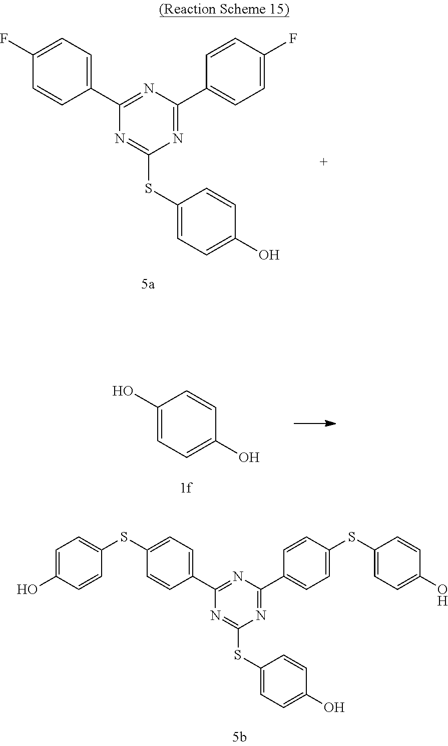 [SMILES: Oc1ccc(Sc2nc(-c3ccc(F)cc3)nc(-c3ccc(F)cc3)n2)cc1 ; Oc1ccc(O)cc1 ; Oc1ccc(Sc2ccc(-c3nc(Sc4ccc(O)cc4)nc(-c4ccc(Sc5ccc(O)cc5)cc4)n3)cc2)cc1]

The compound 5b was synthesized in the same manner as the method of synthesizing the compound 2c except that the compound 5a was used instead of the compound 2b.

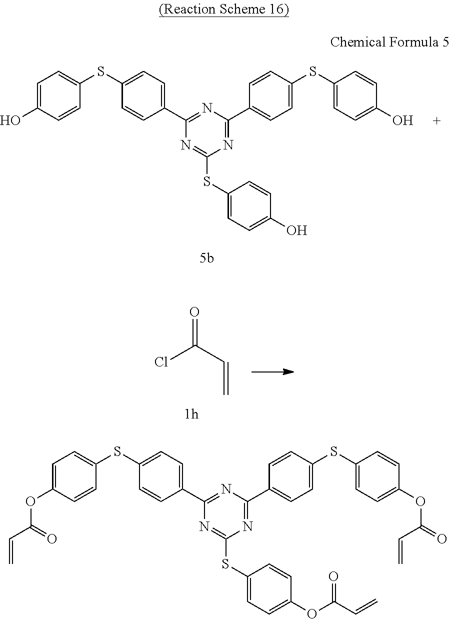

A compound represented by Chemical Formula 5 was synthesized in the same manner as the method of synthesizing the material corresponding to Chemical Formula 1 except that the compound 5b was used instead of the compound 1g.

The analysis result of the compound represented by Chemical Formula 5 is as follows.

MALDI-TOF MS: 768.12 m/z

### Comparative Preparation Example 1: Synthesis of Compound Represented by Chemical Formula C-1

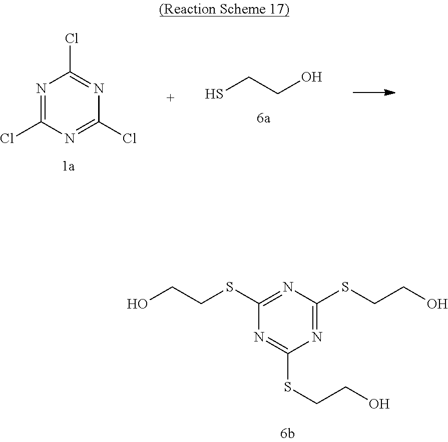

The compound 1a (10.0 mmol) and potassium carbonate (K₂CO₃, 20.0 mmol) were added to 35.0 mL of THF and then cooled to 0° C. The compound 6a (30.0 mmol) was added thereto in a dropwise fashion and then, stirred at 80° C. overnight. After a reaction was completed, distilled water was added thereto to produce precipitates. The solids were separated by filtering under a reduced pressure, washed with distilled water, and dried in a vacuum oven overnight to obtain the compound 6b.

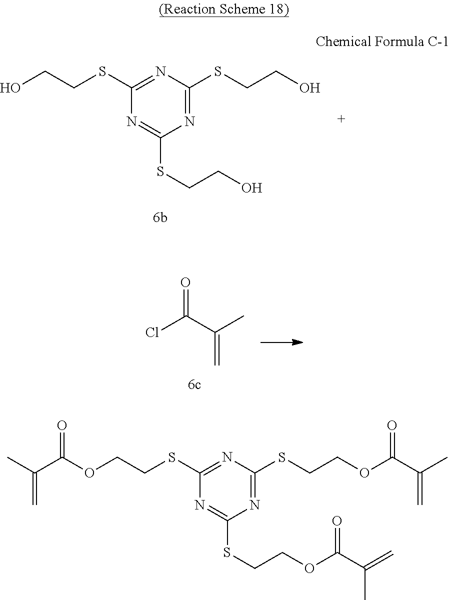

A compound represented by Chemical Formula C-1 was synthesized in the same manner as the method of synthesizing the material corresponding to Chemical Formula 1 except that the compound 6b was used instead of the compound 1g

The analysis result of the compound represented by Chemical Formula C-1 is as follows.

MALDI-TOF MS: 514.11 m/z

### Comparative Preparation Example 2: Synthesis of Compound Represented by Chemical Formula C-2

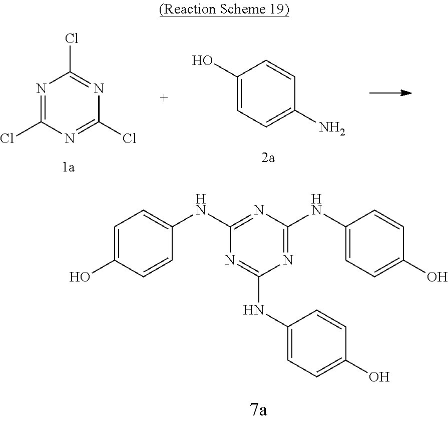 [SMILES: Clc1nc(Cl)nc(Cl)n1 ; Nc1ccc(O)cc1 ; Oc1ccc(Nc2nc(Nc3ccc(O)cc3)nc(Nc3ccc(O)cc3)n2)cc1]

The compound 7a was synthesized in the same manner as the method of synthesizing the compound 6b except that the compound 2a was used instead of the compound 6a.

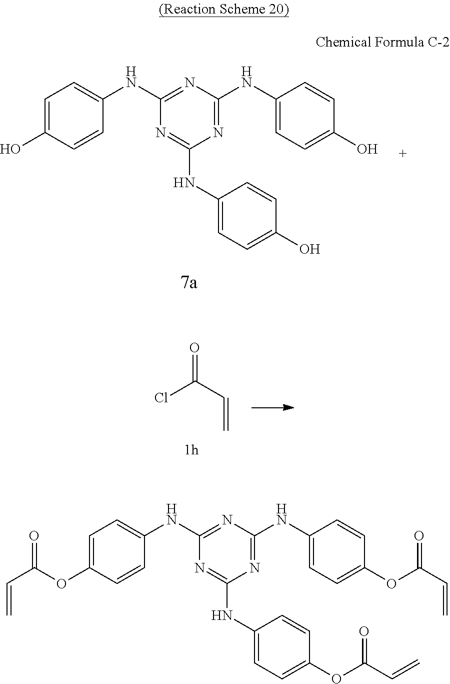

A compound represented by Chemical Formula C-2 was synthesized in the same manner as the method of synthesizing the material corresponding to Chemical Formula 1 except that the compound 7a was used instead of the compound 1g.

The analysis result of the compound represented by Chemical Formula C-2 is as follows.

MALDI-TOF MS: 565.18 m/z

### (Preparation of Photosensitive Resin Compositions)

### Examples 1 to 5 and Comparative Examples 1 and 2

According to the compositions shown in Table 1, the photopolymerization initiator was dissolved in a solvent and stirred at ambient temperature for 2 hours. Then a polymer resin and photopolymerizable monomer were added, stirred at ambient temperature (25° C.) for 1 hour, and then the entire solution was stirred again for 2 hours. The solution was filtered three times through a 0.45 μm filter to remove impurities, thereby producing each photosensitive resin composition.

TABLE 1
(unit: wt %)

| | | Example | Example | Example | Example | Example | Comparative | Comparative |
|---|---|---|---|---|---|---|---|---|
| | | 1 | 2 | 3 | 4 | 5 | Example 1 | Example 2 |
| (A) Binder resin | | 17.5 | 17.5 | 17.5 | 17.5 | 17.5 | 17.5 | 17.5 |
| (B) | (B-1) | 7 | — | — | — | — | — | — |
| Photopolymerizable | (B-2) | — | 7 | — | — | — | — | — |
| monomer | (B-3) | — | — | 7 | — | — | — | — |
| | (B-4) | — | — | — | 7 | — | — | — |
| | (B-5) | — | — | — | — | 7 | — | — |
| | (B-6) | — | — | — | — | — | 7 | — |
| | (B-7) | — | — | — | — | — | — | 7 |

| | | | | | | | |
|---|---|---|---|---|---|---|---|
| (C) Photopolymerization | 1 | 1 | 1 | 1 | 1 | 1 | 1 |
| initiator | | | | | | | |
| (D) Solvent | 74.5 | 74.5 | 74.5 | 74.5 | 74.5 | 74.5 | 74.5 |

(A) Binder resin
Acrylic binder resin (SP-RY38; Resonac Corporation)
(B) Photopolymerizable monomer
(B-1) Photopolymerizable monomer of Preparation Example 1
(B-2) Photopolymerizable monomer of Preparation Example 2
(B-3) Photopolymerizable monomer of Preparation Example 3
(B-4) Photopolymerizable monomer of Preparation Example 4
(B-5) Photopolymerizable monomer of Preparation Example 5
(B-6) Photopolymerizable monomer of Comparative Preparation Example 1
(B-7) Photopolymerizable monomer of Comparative Preparation Example 2
(C) Photopolymerization initiator
Oxime initiator (SPI-03, Samyang Corporation)
(D) Solvent

### Propylene Glycol Monomethyl Ether Acetate (PGMEA, Daicel Corporation)

### EVALUATION

After confirming that the photosensitive resin compositions according to Examples 1 to 5 and Comparative Examples 1 and 2 were all transparent, their refractive indices at 550 nm were measured. Thereafter, each of these was coated on an 8-inch silicon wafer using a SEMES K-SPIN at an rpm capable of exhibiting a similar thickness, followed by soft baking on a 100° C. hot plate, and pattern exposure was performed under the exposure conditions of (Dose: 200 ms/Focus: −0.3) on a i-line stepper made by Nikon Corp. In the exposure process, after measuring a thickness by using TENCOR, the coated substrate was developed to reveal the patterns. Herein, an EHD-100S solution (TMAH) was used as a developer, and time (seconds) (BP) taken for the patterns to appear was measured.

The patterns on the substrate completed with the development were checked with respect to sensitivity and residues through CD-SEM made by Hitachi, Ltd. The pattern was confirmed to be a 96 μm negative pattern, and a large area of residue was confirmed using an Olympus optical microscope, the results are shown in Table 2. The evaluation criteria for developability and pattern linearity are shown in Table 3.

TABLE 2

| | Example | Example | Example | Example | Example | Comparative | Comparative |
|---|---|---|---|---|---|---|---|
| | 1 | 2 | 3 | 4 | 5 | Example 1 | Example 2 |
| Refractive index | 1.727 | 1.715 | 1.711 | 1.702 | 1.698 | 1.51 | 1.56 |
| Development time | 10 | 10 | 10 | 10 | 10 | Undeveloped | Undeveloped |
| (seconds) | | | | | | | |
| Developability | ◯ | ◯ | ◯ | ◯ | ◯ | X | X |
| Pattern linearity | ⊚ | ⊚ | ⊚ | ⊚ | ⊚ | X | X |

TABLE 3

| | Developability | Pattern linearity |
|---|---|---|
| ⊚ | No residue | Very good linearity |
| ◯ | Only slight residue is present | Excellent linearity |
| | around the pattern | |
| Δ | All region residues occur | Lack of linearity |
| X | Undeveloped | Very inferior linearity |

Through the above evaluation, it can be seen that the photosensitive resin composition according to some example embodiments is a transparent photosensitive resin composition, which not only has a very high refractive index at 550 nm, but also has excellent pattern linearity and residue characteristics even with low-temperature curing, making it suitable for use as a microlens surrounding a color filter in a micro OLED.

By way of summation and review, in the micro OLED display panels which may have 10 times smaller pixels than general OLED display panels, red (R)/green (G)/blue (B) light emitting layers may be difficult to form using conventional FMM (Fine Metal Mask) technology. In other words, if the conventional liquid crystal displays are applied to devices for VR, AR, and the like, the color filters may have too large a pattern size to increase resolution.

Accordingly, OLEDoS (OLED on Silicon) technology has recently been introduced to achieve a high resolution of about 4000 ppi or more. The corresponding technology uses OLED deposited on a silicon wafer as a backlight to pattern color filters thereon. While some color filters used for liquid crystal displays may be formed by mounting about 100 μm patterns on a glass and curing it through an exposure process and a post-baking process at a high temperature of about 230° C. or more, the color filters mounted on OLEDoS may not go through the high temperature process due to the OLEDs and should be cured at a low temperature. Additionally, the efficiency of the light emitted from the OLED may be low if it is emitted to the outside. Accordingly, research is continuing on materials with high refractive index that can be cured at low temperatures and are transparent.

Some example embodiments provide a photosensitive resin composition that is transparent and has a high refractive index and is sufficiently cured even at low temperatures.

A photosensitive resin composition according to some example embodiments may be a transparent photosensitive resin composition that can be cured at a low temperature of 100° C. or lower, may have a high refractive index at 550 nm, and may have high transmittance in the visible light range (400 nm to 700 nm), and may be able to achieve the aforementioned effect even with a pre-bake temperature of 100° C. or lower and only photocuring (i-line exposure), so that it may be suitably applied as a micro lens layer (microlens array) in a micro OLED display device capable of implementing a fine pattern.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A photosensitive resin composition, comprising:
a binder resin;
a photopolymerizable monomer;
a photopolymerization initiator; and
a solvent,
wherein:
the photopolymerizable monomer is represented by Chemical Formula 1, 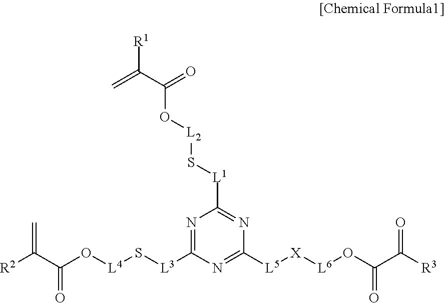
in Chemical Formula 1,
R¹ to R³ are each independently a hydrogen atom or a substituted or unsubstituted C1 to C20 alkyl group,
L¹ to L⁴ are each independently a substituted or unsubstituted C6 to C20 arylene group,
L⁵ is a single bond or a substituted or unsubstituted C6 to C20 arylene group,
L⁶ is a substituted or unsubstituted C1 to C20 alkylene group or a substituted or unsubstituted C6 to C20 arylene group,
X is a single bond, *—S—* or *—NR′—*, and
R′ is a hydrogen atom or a C1 to C10 alkyl group.

2. The photosensitive resin composition as claimed in claim 1, wherein L¹ to L⁴ are all the same.

3. The photosensitive resin composition as claimed in claim 1, wherein R¹ to R³ are all the same.

4. The photosensitive resin composition as claimed in claim 1, wherein:
L⁵ is a substituted or unsubstituted C6 to C20 arylene group, and
X is a single bond or *—S—*.

5. The photosensitive resin composition as claimed in claim 1, wherein:
L⁵ is a single bond and X is *—S—* or *—NR′—*, and
R′ is a hydrogen atom or a C1 to C10 alkyl group.

6. The photosensitive resin composition as claimed in claim 1, wherein L⁵ is a single bond and X is a single bond.

7. The photosensitive resin composition as claimed in claim 1, wherein the photopolymerizable monomer is represented by one of Chemical Formula 1-1 to Chemical Formula 1-5:

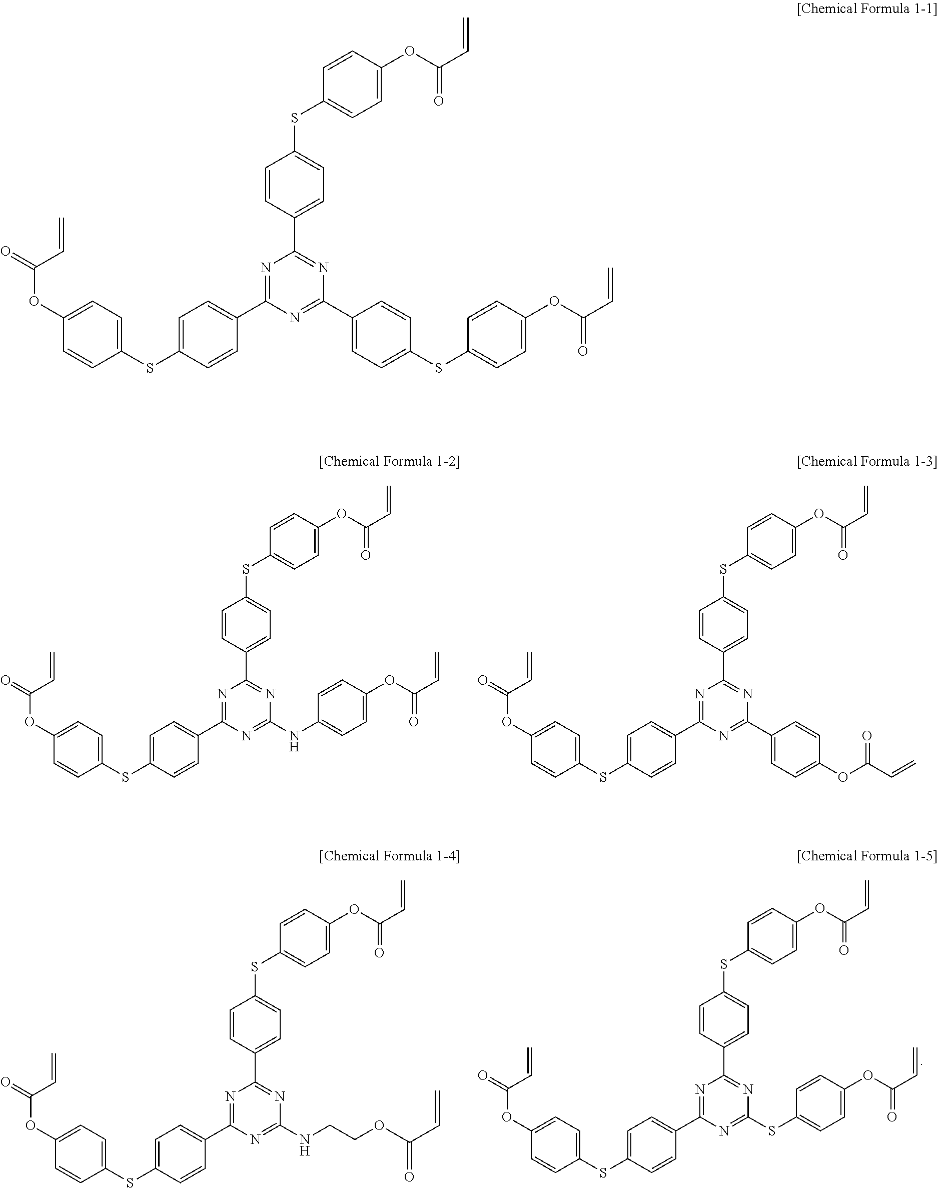 [SMILES: C=CC(=O)Oc1ccc(Sc2ccc(-c3nc(-c4ccc(Sc5ccc(OC(=O)C=C)cc5)cc4)nc(-c4ccc(Sc5ccc(OC(=O)C=C)cc5)cc4)n3)cc2)cc1 ; C=CC(=O)Oc1ccc(Nc2nc(-c3ccc(Sc4ccc(OC(=O)C=C)cc4)cc3)nc(-c3ccc(Sc4ccc(OC(=O)C=C)cc4)cc3)n2)cc1 ; C=CC(=O)Oc1ccc(Sc2ccc(-c3nc(-c4ccc(OC(=O)C=C)cc4)nc(-c4ccc(Sc5ccc(OC(=O)C=C)cc5)cc4)n3)cc2)cc1 ; C=CC(=O)OCCNc1nc(-c2ccc(Sc3ccc(OC(=O)C=C)cc3)cc2)nc(-c2ccc(Sc3ccc(OC(=O)C=C)cc3)cc2)n1 ; C=CC(=O)Oc1ccc(Sc2ccc(-c3nc(Sc4ccc(OC(=O)C=C)cc4)nc(-c4ccc(Sc5ccc(OC(=O)C=C)cc5)cc4)n3)cc2)cc1]

8. The photosensitive resin composition as claimed in claim 1, wherein the photosensitive resin composition includes, based on a total weight of the photosensitive resin composition:
about 10 wt % to about 30 wt % of the binder resin;
about 3 wt % to about 15 wt % of the photopolymerizable monomer; and
about 0.1 wt % to about 5 wt % of the photopolymerization initiator.

9. The photosensitive resin composition as claimed in claim 1, further comprising malonic acid, 3-amino-1,2-propanediol, a silane coupling agent, a leveling agent, a surfactant, a polymerization inhibitor, or a combination thereof.

10. The photosensitive resin composition as claimed in claim 1, wherein the photosensitive resin composition has a refractive index of greater than or equal to about 1.66 at a wavelength of 550 nm.

11. The photosensitive resin composition as claimed in claim 1, wherein the photosensitive resin composition has a transmittance of greater than or equal to about 90% at wavelengths of 400 nm to 700 nm.

12. A photosensitive resin layer manufactured using the photosensitive resin composition as claimed in claim 1.

13. A display device comprising the photosensitive resin layer as claimed in claim 12.

14. The display device as claimed in claim 13, wherein:
the display device is a micro OLED display device including an OLED substrate on a silicon wafer and a color filter layer located on the OLED substrate, the color filter layer being configured to convert white light generated from the OLED substrate into a plurality of color lights,
the photosensitive resin layer is located on the OLED substrate and color filter layer, and
the color filter layer includes a red color filter, a green color filter, and a blue color filter.

15. A method of manufacturing a photosensitive resin layer, the method comprising:
coating the photosensitive resin composition as claimed in claim 1 on a substrate;
prebaking at a temperature of about 100° C. or lower after coating;
exposing to i-line after prebaking; and
developing after exposing.
